(19) United States
(12) Patent Application Publication
(10) Pub. No.: US 2015/0231340 A1
(43) Pub. Date: Aug. 20, 2015

(54) METHODS AND SYSTEMS FOR IDENTIFYING DRY NEBULIZER ELEMENTS

(71) Applicant: NEKTAR THERAPEUTICS, San Francisco, CA (US)

(72) Inventors: Samuel Julius Pumphrey, Cambridge (GB); Montague Fraser Barlow, Cambridge (GB); Andrew Morley, Cambridge (GB); Roger Sewell, Cambridge (GB); Richard Day, Cambridge (GB)

(21) Appl. No.: 14/430,587

(22) PCT Filed: Oct. 17, 2012
(86) PCT No.: PCT/US12/60579

§ 371 Date: Mar. 24, 2015

(51) Int. Cl. A61M 11/00; G01N 27/22

(52) U.S. Cl. CPC A61M 11/005; G01N 27/223; A61M 2205/33; A61M 2205/70; A61M 2205/50

(57) ABSTRACT

Various arrangements for determining an atomization element of a nebulizer is dry are presented. The vibratable element of the nebulizer may be energized with an electrical signal that sweeps from a first frequency to a second frequency. While energizing the vibratable element of the nebulizer with the electrical signal that sweeps from the first frequency to the second frequency, a sequence of impedance values of the vibratable element of the nebulizer may be measured. The sequence of impedance values of the vibratable element of the nebulizer may be analyzed to determine if the atomization element of the nebulizer is wet or dry.

### BACKGROUND

A wide variety of procedures have been proposed to deliver a drug to a patient. In some drug delivery procedures, the drug is a liquid and is dispensed in the form of fine liquid droplets for inhalation by a patient. A patient may inhale the drug for absorption through lung tissue. Such a mist may be formed by a nebulizer. Energizing an element of a nebulizer without a liquid present may result in damage to the nebulizer and/or the nebulizer element.

### SUMMARY

Various arrangements are presented for determining if a nebulizer element is wet or dry. In some embodiments, a nebulizer is presented. The nebulizer may include a nebulizer element comprising an atomization element and a vibratable element. The vibratable element may be configured to vibrate to cause the atomization element to atomize a liquid in contact with the atomization element. The nebulizer may include a reservoir configured to hold the liquid that is to be supplied to the atomization element. The nebulizer may include a control module. The control module may be configured to output an electrical signal at an atomization frequency to energize the vibratable element. The control module may be configured to vary a frequency of the electrical signal across a measurement frequency range to energize the vibratable element. The measurement frequency range may be from a first frequency to a second frequency. While the vibratable element is being energized with the electrical signal that varies from the first frequency to the second frequency, a sequence of impedance values of the vibratable element may be measured by the control module. The control module may analyze the sequence of impedance values to determine if the atomization element is dry.

Embodiments of such a nebulizer may include one or more of the following: The liquid may be a medicament. The control module may be further configured to, if the atomization element is determined to not be in contact with the liquid, cease outputting the electrical signal to energize the vibratable element. The control module being configured to analyze the sequence of impedance values of the vibratable element to determine if the atomization element is dry may comprise the control module being configured to analyze an amount of change among impedance values of the sequence of impedance values. The control module being configured to analyze the sequence of impedance values of the vibratable element to determine if the atomization element is dry may comprise the control module being configured to calculate a sequence of difference values that indicates differences between at least some consecutive impedance values of the sequence of impedance values. The control module being configured to analyze the sequence of impedance values of the vibratable element to determine if the atomization element is dry may comprise the control module being configured to calculate an impedance comparison value using the sequence of difference values and the control module being configured to compare the impedance comparison value to a predefined threshold comparison value to determine if the atomization element is dry.

Additionally or alternatively, embodiments of such a nebulizer may include one or more of the following: The control module being configured to calculate the impedance comparison value using the sequence of difference values may comprise the control module being configured to, for each positive difference value of the sequence of difference values, add a squared value of the positive difference value to the impedance comparison value and for each negative difference value of the sequence of difference values, add an absolute value of the negative difference value to the impedance comparison value. The first frequency may be lower than the second frequency. The control module being configured to output the electrical signal to energize the vibratable element may comprise the control module being configured to output the electrical signal to energize the vibratable element of the nebulizer at multiple different frequencies between the first frequency and the second frequency. The first frequency may be 95 kHz and the second frequency may be 128 kHz. The control module being configured to output the electrical signal to energize the vibratable element may comprise the electrical signal sweeping from the first frequency to the second frequency for less than 200 ms; and the control module may be configured to measure impedance values for the sequence of impedance values at a sampling interval of less than 5 ms. The nebulizer may include a power supply configured to supply the control module with power. The nebulizer may include a mouthpiece configured to allow a person to inhale the liquid atomized by the atomization element. The nebulizer may include a housing configured to couple the nebulizer element with the reservoir.

In some embodiments, a system comprising the nebulizer is presented. The system may include a test module configured to energize the vibratable element while the atomization element is dry with a test electrical signal that sweeps a first frequency range, wherein the measurement frequency range defined by the first frequency and the second frequency is within the first frequency range and is smaller in bandwidth than the first frequency range. The test module may be further configured to, while energizing the vibratable element with the test electrical signal that sweeps the first frequency range, measure a test sequence of impedance values of the vibratable element. The test module may be further configured to determine the first frequency and the second frequency at least partially based on the test sequence of impedance values. The control module of the nebulizer may be further configured to store indications of the first frequency and the second frequency determined by the test module.

In some embodiments, a method for determining an atomization element of a nebulizer is dry may be presented. The method may include energizing a vibratable element of the nebulizer with an electrical signal that sweeps from a first frequency to a second frequency. The method may include, while energizing the vibratable element of the nebulizer with the electrical signal that varies from the first frequency to the second frequency, measuring a sequence of impedance values of the vibratable element of the nebulizer. The method may include analyzing the sequence of impedance values of the vibratable element of the nebulizer to determine if the atomization element of the nebulizer is dry.

Embodiments of such a method may include one or more of the following: The method may include energizing the vibratable element of the nebulizer at an atomization frequency to cause the atomization element to atomize liquid. The liquid may be a medicament. The method may include, if the atomization element is determined to not be in contact with the liquid, cease energizing the vibratable element with the electrical signal. Analyzing the sequence of impedance values of the vibratable element of the nebulizer to determine if the atomization element of the nebulizer is dry may comprise analyzing an amount of change among impedance values of the sequence of impedance values. Analyzing the sequence of impedance values of the vibratable element of the nebulizer to determine if the atomization element is dry may comprise calculating a sequence of difference values that indicates differences between at least some consecutive impedance values of the sequence of impedance values. Analyzing the sequence of impedance values of the vibratable element of the nebulizer to determine if the atomization element of the nebulizer is dry may comprise calculating an impedance comparison value using the sequence of difference values; and comparing the impedance comparison value to a predefined threshold comparison value to determine if the atomization element is wet or dry. Calculating the impedance comparison value using the sequence of difference values may comprise: for each positive difference value of the sequence of difference values, adding a squared value of the positive difference value to the impedance comparison value; and for each negative difference value of the sequence of difference values, adding an absolute value of the negative difference value to the impedance comparison value. The first frequency may be lower than the second frequency. Energizing the vibratable element of the nebulizer with the electrical signal that sweeps from the first frequency to the second frequency may comprise energizing the vibratable element of the nebulizer with the electrical signal at multiple different frequencies between the first frequency and the second frequency. The first frequency may be approximately 95 kHz and the second frequency may be approximately 128 kHz.

Embodiments of such a method may include one or more of the following: The method may include, after ceasing to energize the vibratable element with the electrical signal, waiting a period of time. The method may include, after the period of time, energizing the vibratable element of the nebulizer with the electrical signal that sweeps from the first frequency to the second frequency. The method may also include, after the period of time, while energizing the vibratable element of the nebulizer with the electrical signal that varies from the first frequency to the second frequency, measuring a second sequence of impedance values of the vibratable element of the nebulizer. The method may include, after the period of time, analyzing the second sequence of impedance values of the vibratable element of the nebulizer to determine if the atomization element of the nebulizer is dry. Energizing the vibratable element of the nebulizer with the electrical signal that sweeps from the first frequency to the second frequency may occurs for less than 200 ms. Impedance values for the sequence of impedance values may be measured approximately at a sampling interval of less than 5 ms. The method may be performed at periodic intervals while a liquid is being atomized using the atomization element of the nebulizer. Consecutive periodic intervals of the periodic intervals may be less than two seconds apart. The method may include energizing the vibratable element while dry with a test electrical signal that sweeps a first frequency range, wherein a second frequency range defined by the first frequency and the second frequency is within the first frequency range and is smaller in bandwidth than the first frequency range. The method may include, while energizing the vibratable element with the test electrical signal that sweeps the first frequency range, measuring a test sequence of impedance values of the vibratable element of the nebulizer. The method may include determining the first frequency and the second frequency at least partially based on the test sequence of impedance values.

In some embodiments, an apparatus for determining an atomization element of a nebulizer is dry may be presented. The apparatus may include means for energizing a vibratable element of the nebulizer with an electrical signal that sweeps from a first frequency to a second frequency. The apparatus may include means for measuring a sequence of impedance values of the vibratable element of the nebulizer while energizing the vibratable element of the nebulizer with the electrical signal that sweeps from the first frequency to the second frequency. The apparatus may include means for analyzing the sequence of impedance values of the vibratable element of the nebulizer to determine if the atomization element of the nebulizer is dry.

Embodiments of such an apparatus may include one or more of the following: The apparatus may include means for energizing the vibratable element of the nebulizer at an atomization frequency to cause the atomization element to atomize a liquid. The liquid may be a medicament. The apparatus may include means for ceasing to energize the vibratable element with the electrical signal if the atomization element is determined to not be in contact with the liquid.

In some embodiments, a system for determining an atomization element of a nebulizer is dry is presented. The system may include a controller. The controller may be configured to cause an electrical signal at an atomization frequency to energize a vibratable element of the nebulizer to atomize liquid. The controller may be configured to vary the electrical signal at across a measurement frequency range to energize the vibratable element, wherein the electrical signal sweeps from a first frequency to a second frequency. The controller may be configured to, while the vibratable element is being energized with the electrical signal that sweeps from the first frequency to the second frequency, cause a sequence of impedance values of the vibratable element to be measured. The controller may be configured to analyze the sequence of impedance values to determine if the atomization element is dry.

Embodiments of such a system may include one or more of the following: The liquid may be a medicament. The controller may be further configured to, if the atomization element is determined to not be in contact with the liquid, cease causing the electrical signal to energize the vibratable element. The controller being configured to analyze the sequence of impedance values of the vibratable element of the nebulizer to determine if the atomization element of the nebulizer is dry may comprise the controller being configured to analyze an amount of change among impedance values of the sequence of impedance values.

In some embodiments, a method for delivering a medicament to a patient is presented. The method may include providing a nebulizer comprising a housing defining a mouthpiece and having an atomization element and a vibratable element. The method may include supplying a liquid medicament to the atomization element. The method may include energizing the vibratable element of the nebulizer with an electrical signal at an atomization frequency causing the atomization element to atomize the liquid medicament. The atomized liquid medicament may be available for inhalation through the mouthpiece. The method may include varying the electrical signal across a measurement frequency range that sweeps from a first frequency to a second frequency. The method may include, while sweeping the electrical signal from the first frequency to the second frequency, measuring a sequence of impedance values of the vibratable element of the nebulizer. The method may include analyzing the sequence of impedance values of the vibratable element of the nebulizer to determine the atomization element is dry of the liquid medicament. The method may include ceasing to energize the vibratable element with the electrical signal at least partially based on determining the atomization element is dry of the liquid medicament.

### BRIEF DESCRIPTION OF THE DRAWINGS

A further understanding of the nature and advantages of the present invention may be realized by reference to the following drawings.

FIG. 1 illustrates an embodiment of a nebulizer.

FIG. 2 illustrates an embodiment of a nebulizer driven by a control module.

FIG. 3 illustrates a graph of impedances of embodiments of a nebulizer element energized at various frequencies when wet and dry.

FIG. 4 illustrates an embodiment of a method for determining when an element of a nebulizer is dry.

FIG. 5 illustrates another embodiment of a method for determining when an element of a nebulizer is dry.

FIG. 6 illustrates an embodiment of a method for tailoring a frequency range to a specific nebulizer element and using the tailored frequency range to determine when the element of the nebulizer is dry.

FIG. 7 illustrates an embodiment of a computer system.

### DETAILED DESCRIPTION

Operation of a nebulizer without a liquid present on the nebulizer's element may result in damage to the nebulizer and/or the nebulizer's element. As such, it may be desirable to avoid energizing a nebulizer's element when the element is dry. Various implementations are described for determining whether a nebulizer element is in contact with a liquid (the nebulizer element is wet) or is not in contact with a liquid (the nebulizer element is dry).

Embodiments presented herein are directed to measuring the impedance of a nebulizer element. The impedance of the nebulizer element may be measured periodically and at multiple frequencies. The measured impedance values may be used to determine whether the nebulizer element is in contact with a liquid or not. By measuring the impedance of a nebulizer element across a range of frequencies, it may be determined whether a liquid is in contact with the nebulizer element. It should be understood that in addition to measuring the impedance of the nebulizer element, phase of the nebulizer element may additionally or alternatively be measured and used for determining if the nebulizer element is in contact with a liquid.

A nebulizer element may refer to a component of a nebulizer that vibrates and/or atomizes liquid. A nebulizer element may comprise an atomization element, which atomizes liquid. A nebulizer element may comprise a vibratable element, which, when energized, may vibrate (e.g., expand and contract). When excited at an atomization frequency, the vibratable element may cause the atomization element to vibrate and atomize liquid.

Periodically, a nebulizer element (or, more specifically, the vibratable element of the nebulizer element) may be energized by an electrical signal across a plurality of frequencies (referred to as a “chirp”). This electrical signal may sweep (or step) from a first frequency to a second frequency, such as from a low frequency to a high frequency. While the electrical signal is energizing the nebulizer element, the impedance of the nebulizer element (e.g., the vibratable element) may be measured. Determining the impedance of the nebulizer element may involve taking multiple impedance measurements. Accordingly, multiple, tens, hundreds, or thousands of impedance measurements may be made during a chirp being applied to a nebulizer element. These impedance measurements may be used to determine if the nebulizer element (e.g., the atomization element of the nebulizer element) is wet or dry.

To determine if the nebulizer element is wet or dry using the impedance measurements, calculations based on the impedance measurements may be performed. An increase in an amount of impedance measured across the frequency range may be indicative of a dry nebulizer. Therefore, if the impedances measured during the chirp are determined to increase more than a threshold amount, it may be determined the nebulizer element is dry. Each impedance measurement may be compared with a previous impedance measurement at a lower frequency. If the impedance increases, the difference between the two impedance measurements may be squared and added to an impedance comparison value. If the impedance decreases, the absolute value of the difference may be added to the impedance comparison value. Such calculations may be performed using some or all impedance measurements collected during a chirp. Because the difference value is squared when the impedance is increased, the impedance comparison value will be greater when impedance values tend to increase during the chirp. After some or all of the impedance measurements have been used to compute the impedance comparison value, the impedance comparison value may be compared to a pre-defined threshold comparison value. This comparison with the threshold comparison value may be used to determine if the nebulizer element is wet or dry: if the impedance comparison value is above the threshold comparison value, the nebulizer element may be considered dry; if the impedance comparison value is below the threshold comparison value, the nebulizer element is considered wet.

Such a calculation may be performed periodically, such as once every two seconds, by applying the same chirp (that is, energizing the element by sweeping across the same frequency range), measuring the impedances, calculating the impedance comparison value, and performing the comparison to the threshold comparison value. This may prevent the nebulizer element from operating dry for more than two seconds. If the nebulizer element is determined to be dry, the nebulizer may enter a powered down mode such that the nebulizer element is no longer energized to atomize a liquid. After a period of time, such as several seconds or minutes, another measurement may be performed to confirm the nebulizer element is still dry. If the nebulizer element is still dry, it may be determined all the liquid is exhausted and the nebulizer may remain in the powered down mode. If the nebulizer is determined to be wet (for example, the previous dry determination may have been due to one or more air bubbles being present on the nebulizer element), the nebulizer element may resume being energized to atomize the liquid.

There are various situations where a nebulizer element may potentially be inadvertently operated dry. Such situations, if the nebulizer element is not stopped from being energized, may result in damage to the nebulizer and/or the nebulizer element. For example, a liquid (such as a liquid drug, such as Amikacin) may have previously been in contact with a nebulizer element, but the supply of liquid may have become exhausted. A particular dose of such a liquid drug may be provided to a nebulizer element to be atomized for delivery to a patient. At the end of the dose, the nebulizer element may inadvertently continue to be energized although the entire dose of the liquid drug has been atomized, thus resulting in a dry nebulizer element being energized. As another example, a nebulizer element may inadvertently be energized without any liquid being in contact with the nebulizer element. In both of these instances, the nebulizer and/or its element may be damaged by being energized while dry. Other situations also exist where it may be beneficial to identify a dry nebulizer element.

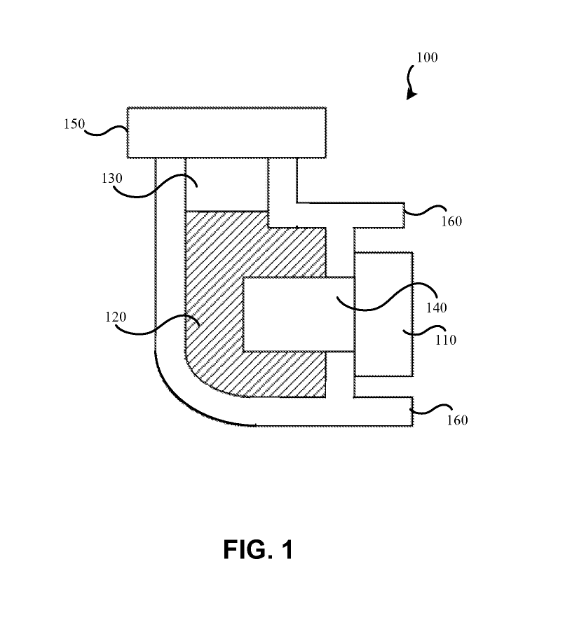

FIG. 1 illustrates an embodiment of a nebulizer 100. The nebulizer 100 may include nebulizer element 110, drug reservoir 120, head space 130, interface 140, and cap 150. Nebulizer element 110 may be comprised of a vibratable element (e.g., a piezoelectric ring), that expands and contracts when an electric signal is applied. The vibratable element may be attached to an atomization element (e.g., a perforated membrane), which may be part of nebulizer element 110. An electrical signal applied to nebulizer element 110 may pass through only the vibratable element (e.g, piezoelectric ring). The atomization element coupled with the vibratable element may affect the impedance of the vibratable element. The atomization element may be a perforated membrane and may have a number of holes passing through it. When an electrical signal is applied to the vibratable element (e.g., the piezoelectric ring), this may cause the atomization element (e.g., the perforated membrane) to move and/or flex (e.g., vibrate). Such movement of the atomization element while in contact with a liquid may cause the liquid to atomize, generating a mist of liquid particles. In some embodiments, the atomization element of nebulizer element 110 may include an aperture plate.

A supply of a liquid, commonly a liquid drug (examples of which are detailed later in this document), may be stored in the drug reservoir 120. As illustrated in FIG. 1, drug reservoir 120 is only partially filled with the liquid drug. A housing may be used to couple drug reservoir 120 to nebulizer element 110. The housing may define a mouthpiece that can be used by a person to inhale atomized liquid drug. As the liquid drug is atomized, the amount of the liquid drug remaining in drug reservoir 120 may decrease. Depending on the amount of the liquid drug in the drug reservoir 120, only a portion of the reservoir may be filled with the liquid drug. The remaining portion of drug reservoir 120 may be filled with gas, such as air. This space maybe referred to as head space 130. An interface 140 may serve to transfer the liquid drug between drug reservoir 120 and nebulizer element 110. A mouthpiece 160 may be present to serve as an interface between a patient's mouth and the nebulizer. Nebulizer element 110 may deliver atomized liquid to mouthpiece 160, which a patient may hold in his or her mouth.

Nebulizers, and the techniques associated with nebulizers, are described generally in U.S. Pat. Nos. 5,164,740; 5,938,117; 5,586,550; 5,758,637; 6,014,970; 6,085,740; 6,235,177; 6,615,824; and 7,322,349, the complete disclosures of which are incorporated by reference for all purposes.

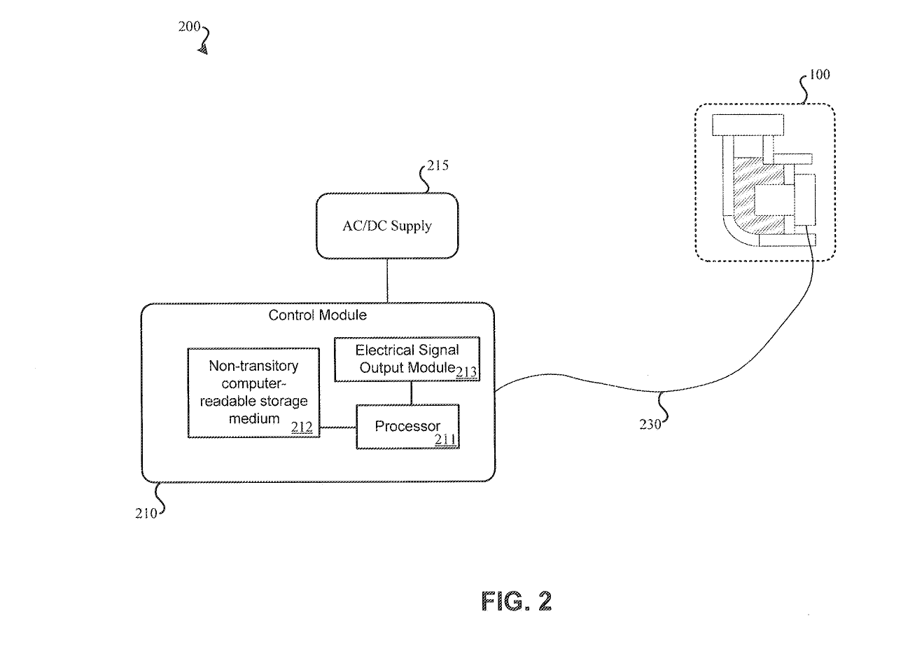

A nebulizer, such as nebulizer 100, may be connected with a control module such as illustrated in FIG. 2. FIG. 2 illustrates a simplified block diagram of an embodiment 200 of a nebulizer control module coupled with nebulizer 100. Nebulizer 100 of FIG. 2, which may represent nebulizer 100 of FIG. 1 or some other nebulizer such as those described in the referenced applications, may be connected with control module 210 via wire 230, which may be a cable. Wire 230 may allow control module 210 to transmit an electrical signal of varying frequency and varying voltage through wire 230 to nebulizer 100. Control module 210 may be connected to voltage supply 215 capable of supplying a DC voltage and/or an AC voltage to control module 210.

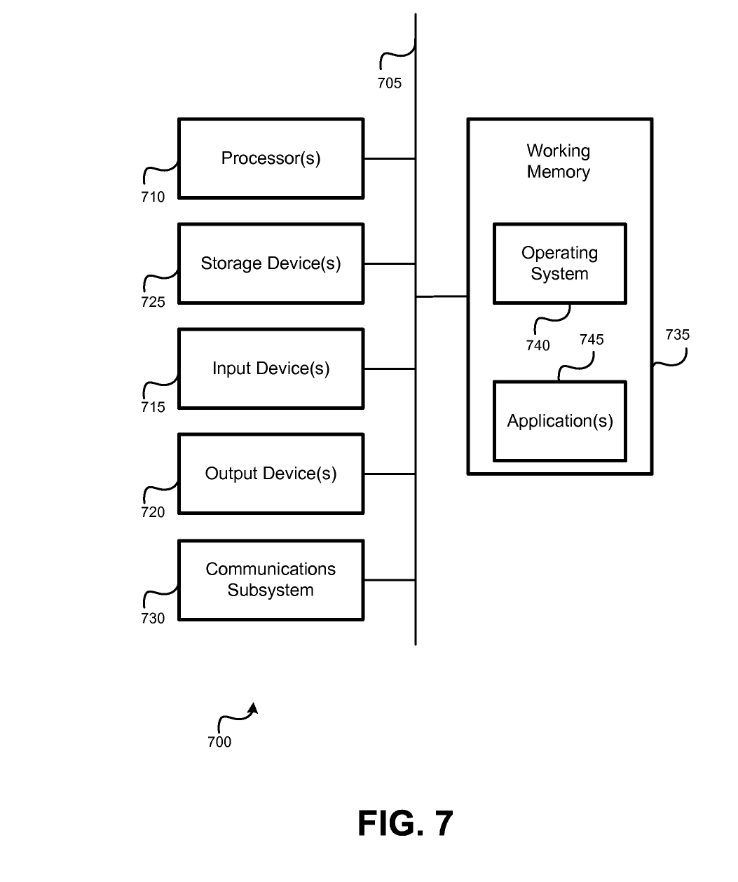

Control module 210 may contain various components. In some embodiments of control module 210, processor 211 (e.g., a controller), non-transitory computer-readable storage medium 212, and electrical signal output module 213 are present. Processor 211 may be a general purpose processor or a processor designed specifically for functioning in control module 210. Processor 211 may serve to execute instructions stored as software or firmware. Such instructions may be stored on non-transitory computer-readable storage medium 212. Non-transitory computer-readable storage medium 212 may be random access memory, flash memory, a hard drive, or some other storage medium capable of storing instructions. Instructions stored by non-transitory computer-readable storage medium 212 may be executed by processor 211, the execution of the instructions resulting in electrical signal output module 213 generating an electrical signal of a varying frequency and/or varying voltage that is output to the nebulizer element of nebulizer 100 via wire 230. In some embodiments, control module 210 may be computerized and may contain a computer system as presented in FIG. 7.

The electrical signal output by electrical signal output module 213 may include one or more frequencies. For example, electrical signal output module 213 may generate an electrical signal that sweeps across or steps (sweeping and stepping may be collectively referred to as varying) across multiple frequencies to energize the nebulizer element. The impedance of the nebulizer element may be measured while one or more frequencies of the electrical signal are being used to energize the nebulizer element. To atomize liquid, an electrical signal at one or more particular frequencies may be output by electrical signal output module 213 to energize the nebulizer element. In some embodiments, multiple frequencies may output by electrical signal output module 213 to energize the nebulizer element.

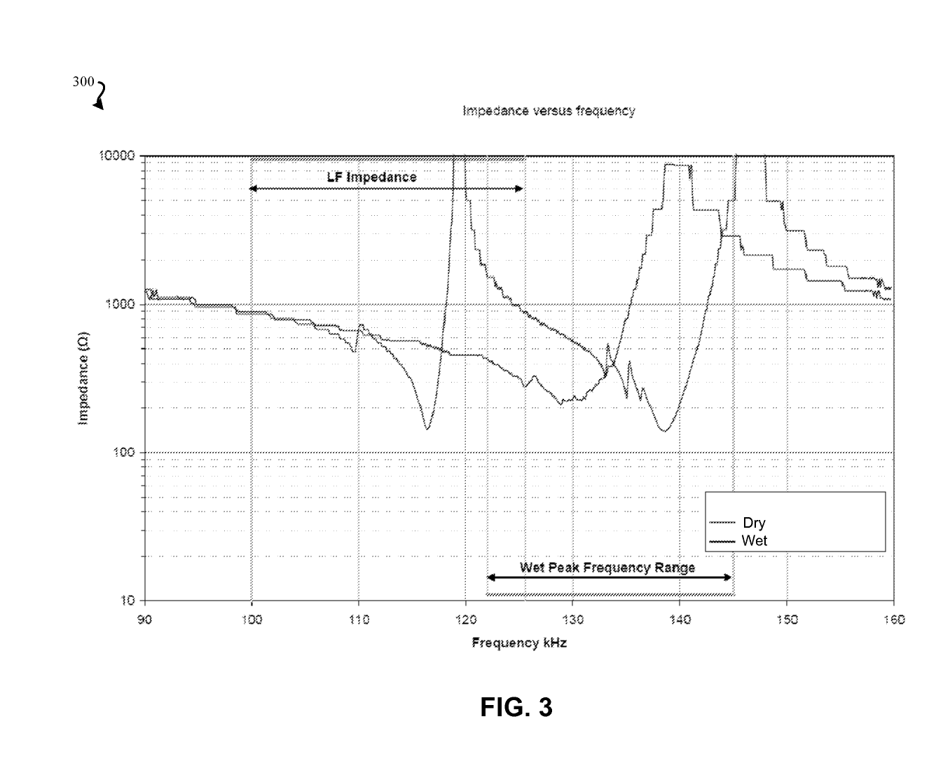

FIG. 3 illustrates a graph 300 of impedances of an embodiment of a nebulizer element when wet and dry at various frequencies. More specifically, the impedances may be of a vibratable element of the nebulizer element. Whether liquid is in contact with a atomization element of a nebulizer element may affect the impedance of the vibratable element of the nebulizer element. As illustrated in graph 300, whether an atomization element is wet or dry may cause the vibratable element's impedance to vary at various frequencies. For example, at approximately 119 kHz, the vibratable element of the nebulizer element has a greater impedance when dry than when wet. Between 100 kHz and 125 kHz, the impedance of the vibratable element when wet trends downward from approximately 900 ohms to 200 ohms with no significant increase in impedance. However, over the same frequency range, impedance of the vibratable element when dry decreases to approximately 150 ohms, spikes to over 10,000 ohms near 119 kHz, and drops to approximately 900 ohms at 125 kHz. As such, a line graphing the impedance of the vibratable element when the atomization element is dry may exhibit, over at least a portion of the frequency range, a positive slope that is greater than the slope of a line graphing the impedance of the vibratable element when the atomization element is wet over the same frequency range. Accordingly, by analyzing the change in impedance of a vibratable element over the frequency range, it may be possible to accurately determine whether the atomization element coupled with the vibratable element is wet or dry.

In graph 300, LF (low frequency) impedance range may indicate the frequency range over which impedances of the vibratable element are measured. Within this frequency range, a dry atomization element may cause a positive increase in impedance (for at least a portion of the frequency range) of the vibratable element, while a wet atomization element may not exhibit a similar positive increase in impedance of the vibratable element. It should be understood that graph 300 illustrates the impedance characteristics of a particular type of nebulizer element (e.g., combination of vibratable element and atomization element). Other types of nebulizer elements may exhibit different impedances in response to being energized at various frequencies. For other types of nebulizer elements, the frequency range over which the vibratable element is energized to determine if the atomization element is wet or dry may be selected based on frequencies where it has been empirically determined (or calculated) that a vibratable element has a significantly different impedance when the atomization element is dry compared to when the atomization element is wet.

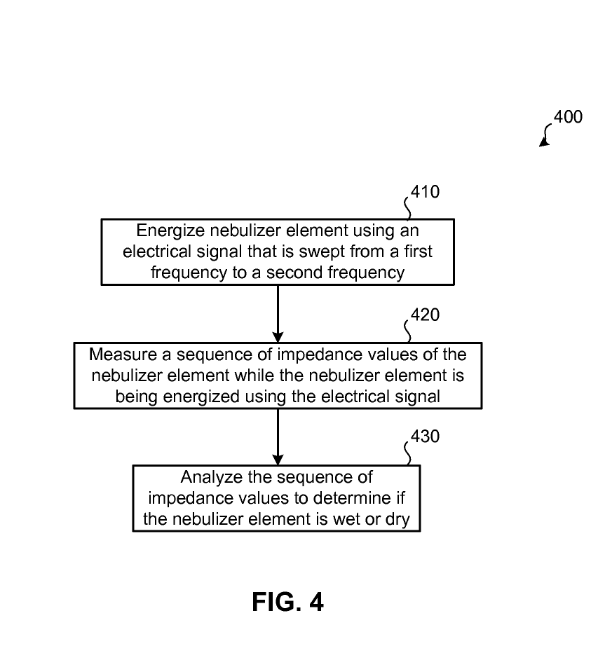

Various methods to determine if a nebulizer element is wet or dry may be performed using the nebulizer 100 of FIG. 1 and/or the control module 210 of FIG. 2. FIG. 4 illustrates an embodiment of a method 400 for determining when an element of a nebulizer is dry. More specifically, method 400 may be used to determine when a atomization element of a nebulizer element is dry. Method 400 may involve using the nebulizer 100 of FIG. 1 and/or the control module 210 of FIG. 2. Means for performing method 400 include a control module, a processor, an electrical signal output module, a computerized device, a nebulizer, a nebulizer element (which may include an atomization element, such as a perforated membrane, and a vibratable element, such as a piezoelectric ring), and a computer-readable storage medium.

At step 410, a nebulizer element of a nebulizer may be energized by one or more electrical signals across a range of frequencies generated by a control module. The characteristics of the nebulizer element when energized may have already been analyzed at various voltages and frequencies, such as the nebulizer element used to produce the graph of FIG. 3. Therefore, it may already be known at what frequency or frequency ranges and/or voltages the impedance of the nebulizer element while wet varies significantly from the impedance of the nebulizer element while dry. For example, if the nebulizer element being used while performing method 400 is the nebulizer element used to create FIG. 3, the nebulizer element may be energized over a frequency range from 95 kHz-128 kHz (which spans 33 kHz in size). This frequency range may have been selected due to the significant differences in the impedance of the nebulizer element when wet compared to dry. To energize the nebulizer element over the frequency range from 95 kHz to 128 kHz, the device producing the electrical signal may start at 95 kHz and sweep up to 128 kHz. In other embodiments, the device producing the electrical signal may start at 128 kHz and sweep down to 95 kHz. It should be understood that in other embodiments other frequency ranges are possible.

Energizing the nebulizer element over a range of frequencies may involve sweeping from a first frequency to a second frequency such that frequencies between the first frequency and the second frequency are used to energize the nebulizer element. In some embodiments, rather than sweeping between two frequencies, stepping between the two frequencies may occur. This may involve the nebulizer element being energized at particular frequencies, each for an amount of time, between the first and second frequencies. Sweeping or stepping (which may be collectively referred to as varying) through a frequency range 33 kHz in size may take a period of time such as 160 ms. Further, it should be understood that the nebulizer element may be energized with multiple, pulsed frequencies at a time.

At step 420, a sequence of impedance values of the nebulizer element may be measured while the nebulizer element is being energized by the electrical signal being swept or stepped through the range of frequencies. As such, while the frequency range is being swept, impedance measurements may be measured. Impedance measurements may be captured at predefined intervals, such as once every millisecond. Therefore, if the period of time over which the frequency range is swept is 160 ms, 160 impedance measurements may be performed. Phase may also be measured at the same or a different interval.

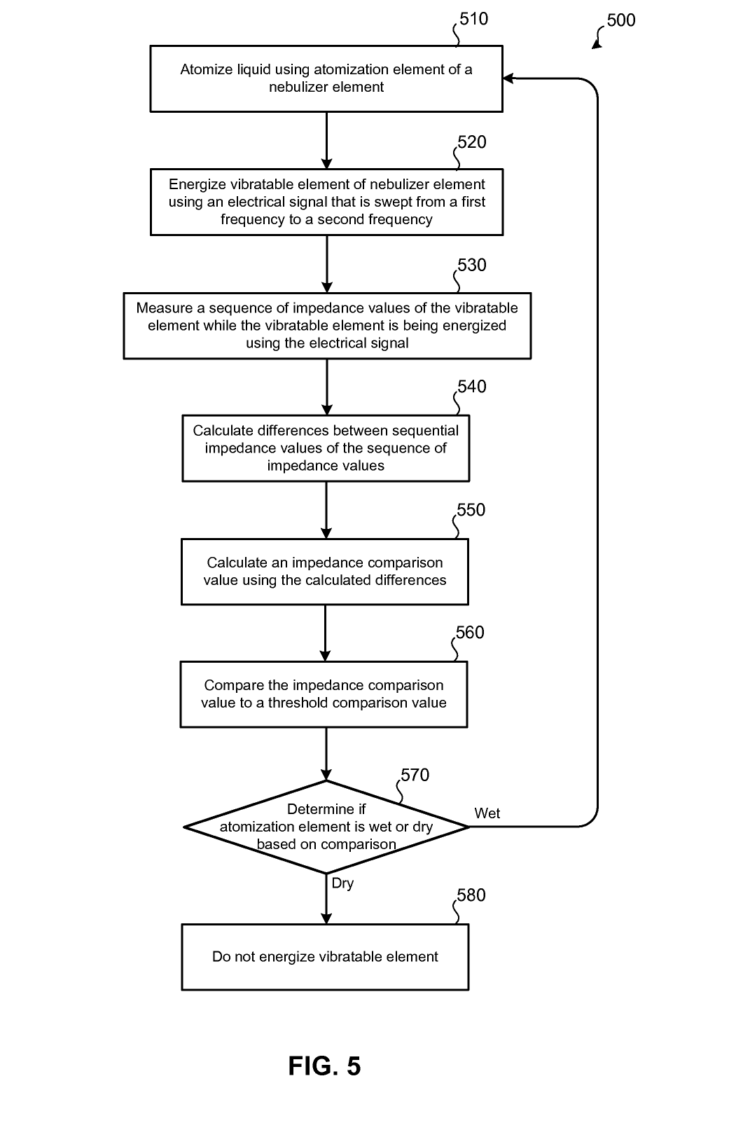

At step 430, the sequence of impedance values measured at step 420 may be used to determine if the nebulizer element is wet or dry. Analyzing the impedance values may involve determining if a positive slope is present among impedance values within the frequency range as illustrated in FIG. 3. It may be possible to determine if such a positive slope is present without producing such a graph. An amount of change among (consecutive) impedance values of the sequence may be analyzed to determine if a positive slope is present. While the methods detailed herein focus on determining if a positive slope is present among impedance values, other embodiments may use the presence of a negative slope to determine if the nebulizer element is wet or dry. Additional details on how such analyzing may be performed is detailed in relation to method 500 of FIG. 5. It should be understood that other metrics may also be used to determine if the nebulizer element is wet or dry. For instance, phase change measurements, absolute impedance measurements, and/or metrics other than the amount of change of impedance over a frequency range may be used.

Following step 430, if the nebulizer element was determined to be wet, the nebulizer element may be energized with one or more frequencies appropriate to atomize a liquid. The nebulizer element may continue to be energized to atomize the liquid for a period of time until method 400 is repeated. For instance, method 400 may be repeated once every 1, 1.6, 2, 3, or 4 seconds, or at some other interval. For example, if method 400 is repeated once every 1.6 seconds, the nebulizer element is unlikely to be energized for more than 1.6 seconds while dry. Reducing the amount of time the nebulizer element may be energized while dry may limit the possibility of damage to the nebulizer element.

FIG. 5 illustrates an embodiment of a method 500 for determining when an element of a nebulizer is dry. More specifically, method 500 may be used to determine when a atomization element, such as a perforated membrane, of a nebulizer element is dry. Method 500 may involve using the nebulizer 100 of FIG. 1 and/or the control module 210 of FIG. 2. Means for performing method 500 include a control module, a processor, an electrical signal output module, a computerized device, a nebulizer, a nebulizer element (which may include a vibratable element and a atomization element), and a computer-readable storage medium. Method 500 may represent a more detailed embodiment of method 400 of FIG. 4.

Initially, a liquid, such as a liquid medicament, may be supplied to a nebulizer. This may be performed by the liquid being added to a reservoir of the nebulizer. From the reservoir, the liquid may be drawn and put in contact with the atomization element of the nebulizer element, this making the atomization element wet with the liquid. At step 510, the vibratable element may be energized by an electrical signal at one or more atomization frequencies. This may result in the atomization element vibrating and atomizing the liquid that is in contact with the atomization element. Step 510 may continue for a predefined period of time. For example, step 510 may be performed for approximately 1.6 seconds before proceeding to step 520. It should be understood that the period of time that step 510 is performed may be configurable. If the atomization element is dry, the atomization element may continue to be energized until the predefined period of time for step 510 expires.

At step 520, the vibratable element may be energized by an electrical signal at a range of frequencies, which may be referred to as a measurement frequency range. These frequencies may be generated by a control module. As such, the electrical signal used to energize the vibratable element at step 510 may be varied to the measurement frequency range of step 520. The measurement frequency range may include the atomization frequency or the atomization frequency may be outside of the measurement frequency range. As such, during step 520 (and other steps of method 500 besides step 510), the vibratable element may not be energized with the electrical signal to cause the atomization element to atomize liquid. The characteristics of the nebulizer element when energized may have already been analyzed at various voltages and frequencies, such as the nebulizer element used to produce the graph of FIG. 3. Therefore, it may already be known at what frequency or frequencies the impedance of the vibratable element while the atomization element is wet has a significantly different impedance than when the atomization element is dry. For example, if the nebulizer element being used while performing method 400 is the nebulizer element used to create FIG. 3, the vibratable element of the nebulizer element may be energized over a frequency range from 95 kHz-128 kHz (which spans 33 kHz). This frequency range may have been selected due to the significant differences in the impedance of the vibratable element when wet compared to dry. To energize the nebulizer element over the frequency range from 95 kHz to 128 kHz, the device producing the electrical signal may start at 95 kHz and sweep up to 128 kHz. In other embodiments, the device producing the electrical signal may start at 128 kHz and sweep down to 95 kHz. It should be understood that in other embodiments (for the same or different nebulizer element) other frequency ranges are possible.

Energizing the vibratable element over a range of frequencies may involve sweeping from a first frequency to a second frequency such that frequencies between the first frequency and the second frequency are used to energize the vibratable element. In some embodiments, rather than sweeping between two frequencies, stepping between the two frequencies may occur. This may involve the vibratable element being energized with particular predefined frequencies between the first and second frequencies. Sweeping or stepping through a frequency range 33 kHz in size may take a period of time such as 160 ms. Further, it should be understood that the nebulizer element may be energized with multiple, pulsed frequencies at a time.

At step 530, a sequence of impedance values of the vibratable element may be measured while the vibratable element is being energized by the electrical signal being swept or stepped through the range of frequencies. As such, while the frequency range is being swept and used to energize the vibratable element, impedance measurements of the vibratable element may be measured. Impedance measurements may be captured at predefined intervals, such as once every millisecond. Therefore, if the period of time over which the frequency range is swept is 160 ms, 160 impedance measurements may be performed. In other embodiments, impedance measurements may be captured at different time intervals. Phase may also be measured at the same or a different interval.

At step 540, the differences between impedance values may be calculated. Each difference value may represent a difference between two consecutive impedance values of the sequence of impedance values; for example, if impedance values are measured every millisecond. A difference between two consecutive impedance values may represent a change in impedance over the millisecond. Equation 1 may be used to calculate the difference values.

ΔΩ(i)=Ω(i)−Ω(i−1) Eq. 1

According to equation 1, a difference value may be obtained by subtracting the previous impedance value (i−1) in the sequence of impedance values from the impedance value (i). Therefore, if impedance values increase between the two values, the difference value will be positive and if impedance values decrease between the two values, the difference value will be negative. In other embodiments, only some of the impedance values measured at step 530 may be used to determine difference values. For instance, every other impedance value of the sequence of impedance values may be used.

At step 550, an impedance comparison value may be calculated using the difference values calculated at step 540. The impedance comparison value may be calculated using all or some of the difference values calculated at step 540. The impedance comparison value may be used for comparison with a threshold value to determine if the atomization element is wet or dry. As shown in FIG. 3, a positive slope within the frequency range applied to the nebulizer element may be indicative of a dry nebulizer element. As such, determining when a positive slope is present (that is, measured impedance values increase as frequency increases) may be desired. To do this, various calculations may be performed. A calculation that may be performed to determine if impedance is increasing involves equations 2 and 3.

Ω_{COMPARISON}=Ω_{COMPARISON}+(ΔΩ(i))² if ΔΩ(i)>0 Eq. 2

Ω_{COMPARISON}=Ω_{COMPARISON}+|ΔΩ(i)| if ΔΩ(i)≦0 Eq. 3

The impedance comparison value (Ω_{COMPARISON}) may initially be set to zero for step 550 and may be a summation that is increased for each difference value calculated at step 540. In some embodiments, each difference value calculated at step 540 may be used to determine a single impedance comparison value; in other embodiments, only some of the difference values may be used. Since when a difference value is positive (indicative of an increase in impedance or a positive slope) the difference value is squared and added to the impedance comparison value, but when the difference value is negative (indicative of a decrease in impedance or a negative slope) only the absolute value of the difference is added to the impedance comparison value, the final impedance comparison value may be expected to be significantly greater when an increase in impedance is present within at least part of the frequency range.

Equations 1 through 3 are examples of how to determine an impedance comparison value that can be used for comparison to a threshold value to determine whether or not a atomization element is wet or dry. It should be understood that other possible ways of determining such an impedance comparison value are possible.

At step 560, the impedance comparison value determined at step 550 may be compared to a threshold comparison value. This threshold comparison value may have been empirically determined. For example, a threshold comparison value may be selected that tends to be greater than impedance comparison values calculated for wet atomization elements, but less than impedance comparison values calculated for dry atomization elements. If an impedance comparison value is less than the threshold comparison value, the atomization element may be likely to be wet. If the impedance comparison value is greater than the threshold comparison value, the atomization element may be likely to be dry.

At step 570, if the comparison of step 560 indicates the impedance comparison value is greater than the threshold value, method 500 may proceed to step 580. At step 580, the vibratable element may stop being energized to atomize liquid. This may be because the atomization element is expected to be dry. If method 500 proceeds to step 580, the vibratable element may not be energized until a user provides an indication that the vibratable element is to be energized again. In some embodiments, a period of time may be waited and the nebulizer element may be reanalyzed to determine if the atomization element is wet or dry. This may be performed to determine if the determination that the atomization element was dry was due to one or more bubbles being present on the atomization element (which may have since dissipated or moved). If the atomization element is subsequently determined to be wet, method 500 may return to step 510. If the atomization element is again determined to be dry, the vibratable element may remain unenergized. At step 570, other measurements may also be used to determine if the vibratable element is wet or dry, such as phase measurements.

At step 570, if the comparison of step 560 indicates the impedance comparison value is less than the threshold value (e.g., the nebulizer element is likely wet), method 500 may return to step 510. At step 510, the vibratable element may be energized at one or more frequencies to cause the atomization element to atomize a liquid, such as a liquid drug for a period of time, before performing the remaining steps of method 500 again. Method 500 may continue to be performed until the atomization element is determined to be dry and the vibratable element no longer energized, either to cause the atomization element to atomize liquid or to determine whether the atomization element is wet or dry.

It has been found that the increase in impedance when the atomization element is dry may vary by nebulizer element, even across nebulizer elements of the same make and model. Referring to the graph of FIG. 3, the graphed vibratable element's impedance increases and peaks around 119 kHz. However, other vibratable elements (which may be made by the same manufacturer and may be the same model) may increase and/or peak at a different frequency. In method 500, a frequency range is swept that is broad enough to encompass a range of frequencies across which it is expected that most nebulizer elements (at least of the same make and model) may be expected to increase (and possibly peak) when the atomization element of the nebulizer element is dry. The bandwidth of the range of frequencies that is swept to determine if the atomization element is wet or dry may be decreased (and thus the amount of time sweeping frequencies may be decreased) by tailoring the frequency range to individual nebulizer elements. Tailoring the frequency range for individual nebulizer elements may be useful in a manufacturing setting, such as to speed testing of a nebulizer's ability to properly identify whether the atomization element is wet or dry. After manufacturing, the tailored frequency range may be stored by the nebulizer for use in decreasing the amount of time spent sweeping frequencies. As such, more time may be spent energizing the vibratable element to cause the atomization element to atomize liquid rather than test for whether the atomization element is wet or dry.

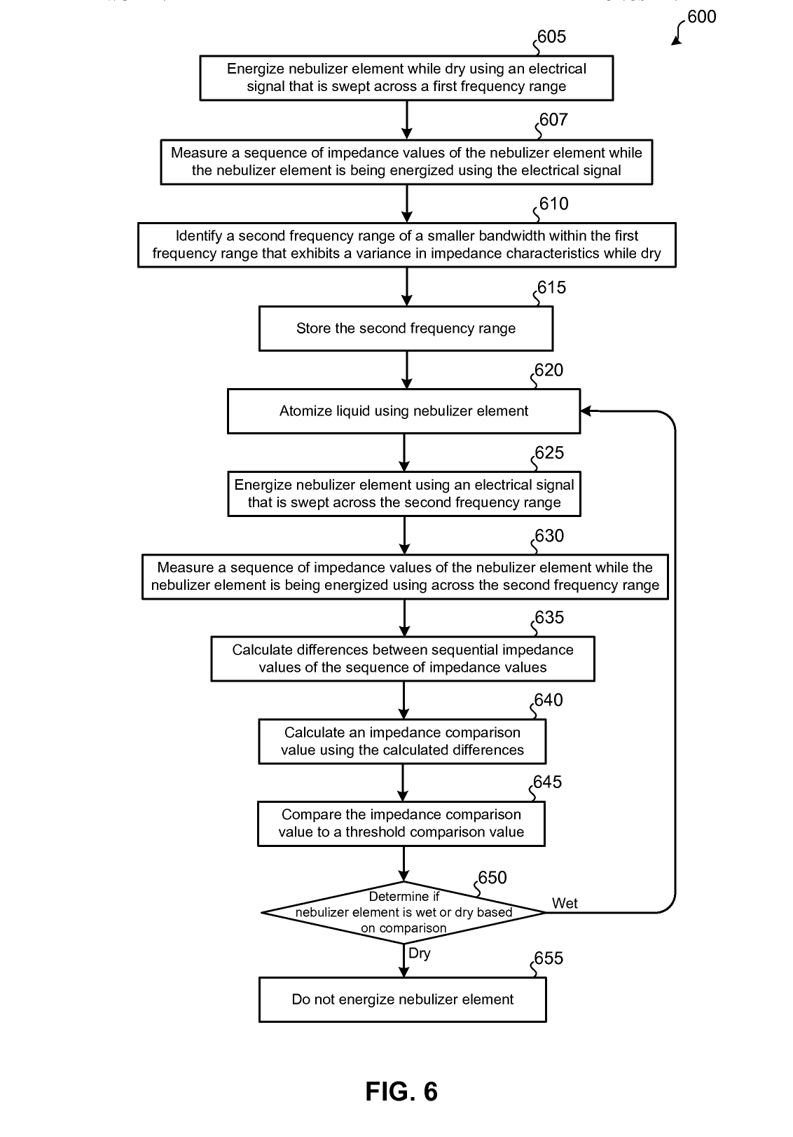

FIG. 6 illustrates an embodiment of a method 600 for tailoring a frequency range to a specific nebulizer element and using the tailored frequency range to determine when the element of the nebulizer is dry. Method 600 may involve using the nebulizer 100 of FIG. 1 and/or the control module 210 of FIG. 2. Means for performing method 600 include a test module, control module, a processor, an electrical signal output module, a computerized device, a nebulizer, a nebulizer element, and a computer-readable storage medium. Method 600 may represent a more detailed embodiment of method 400 of FIG. 4 and/or method 500 of FIG. 5. Further, portions of method 600 may be performed by a test module. Such a test module may be present in a manufacturing environment to test the functionality of the nebulizer and/or determine a tailored frequency range for testing purposes and/or post-manufacturing operation. The test module may be computerized and may contain at least some components similar to a control module, such as control module 210 of FIG. 2. During testing, a test module may perform functions on the nebulizer and nebulizer element that are normally performed by a control module. As such, a test module may be configured to perform at least some same functions as a control module.

At step 605, the vibratable element of the nebulizer element may be energized while the atomization element of the nebulizer element is dry using a test electrical signal that is swept through a first frequency range. Step 605 may be performed by a test module or a control module. While the frequency range over which the impedance of a vibratable element increases and/or peaks is expected to vary for individual nebulizers, the first frequency range may have a sufficient bandwidth that it is likely the vibratable element impedance will increase and/or peak while dry within the first frequency range. For example, the first frequency range may be from 95 kHz to 128 kHz. It should be understood that some other frequency range may be used. At step 607, the impedance of the vibratable element may be measured while the nebulizer element is being energized with the first frequency range of step 607. Each of these impedance measurements may be stored, at least temporarily.

At step 610, the impedance measurements stored at step 607 may be analyzed to determine a second frequency range of smaller bandwidth within the first frequency range over which the impedance of the vibratable element tends to increase. Step 610 may be performed by a test module or a control module. For example, referring to FIG. 3, if the first frequency range is 95 kHz to 128 kHz, the second frequency range may be 115 kHz to 119 kHz. This range of 115 Khz to 119 kHz may work well for use in identifying a dry atomization element for the specific nebulizer element method 600 is being performed on; however, this frequency range may not work well for identifying when the atomization element is dry for other nebulizer elements, even if the other nebulizer elements are of the same make and model.

At step 615, the second frequency range that is of smaller bandwidth than the first frequency range may be stored. This second frequency range may be stored by the test module (e.g., for use during testing) and/or by the control module (e.g., for use after testing, such as during post-manufacturing operation). If the smaller bandwidth frequency range is to be used during normal operation (outside of a manufacturing and test environment), the second frequency range may be stored local to the nebulizer, such as in non-transitory computer-readable storage medium 212 of control module 210. If the smaller bandwidth frequency range is to only be used for an initial test of the nebulizer's ability to detect a wet and dry nebulizer element, the smaller frequency range may be stored to a device (e.g., test equipment) external to the nebulizer.

Between steps 615 and 620, a liquid may be provided and put in contact with the atomization element. At step 620, the vibratable element may be energized by an electrical signal at one or more frequencies to atomize a liquid. Step 620 may continue for a predefined period of time. For example, step 620 may be performed for approximately 1.6 seconds before proceeding to step 625. It should be understood that the period of time that step 620 is performed may be configurable. If the atomization element is dry, the vibratable element may continue to be energized until the predefined period of time for step 620 expires.

At step 625, the vibratable element may be energized by an electrical signal at the second range of frequencies. Step 625 may be performed by a test module or a control module. These frequencies may be generated by a control module or a separate test piece of hardware. The frequencies used at step 625 may be different from the one or more frequencies used at step 615 to atomize the liquid. Since the second frequency range over which the impedance values increase was previously determined at step 610, this smaller bandwidth frequency range may be used to determine if the atomization element is wet or dry. To energize the vibratable element over the second frequency range, the device producing the electrical signal may start at the lower end of the second frequency range and sweep up to the upper end of the second frequency range. In other embodiments, the device producing the electrical signal may start at the upper end of the second frequency range and sweep down to the lower end of the second frequency range.

Energizing the vibratable element over the second range of frequencies may involve sweeping from a first frequency to a second frequency of the second frequency range such that frequencies between the first frequency and the second frequency are used to energize the vibratable element. In some embodiments, rather than sweeping between two frequencies, stepping between the two frequencies may occur. This may involve the vibratable element being energized with particular predefined frequencies between the first and second frequencies. Sweeping or stepping through the second frequency range may take less time than sweeping or stepping through the first frequency range because the second frequency range has a smaller bandwidth.

At step 630, a sequence of impedance values of the vibratable element may be measured while the vibratable element is being energized by the electrical signal being swept or stepped through the second range of frequencies. Step 630 may be performed by a test module or a control module. While the frequency range is being swept and used to energize the element, impedance measurements of the element may be measured. Impedance measurements may be captured at predefined intervals, such as once every millisecond. Therefore, if the period of time over which the frequency range is swept is 50 ms, 50 impedance measurements may be performed. In other embodiments, impedance measurements may be captured at different time intervals. Phase may also be measured at the same or a different interval.

At step 635, the differences between impedance values may be calculated. Step 630 may be performed by a test module or a control module. Each difference value may represent a difference between two consecutive impedance values of the sequence of impedance values; for example, if impedance values are measured every millisecond. A difference between two consecutive impedance values may represent a change in impedance over the millisecond. Equation 1 may be used to calculate the difference values as detailed in relation to method 500.

At step 640, an impedance comparison value may be calculated using the difference values calculated at step 635. Step 640 may be performed by a test module or a control module. The impedance comparison value may be calculated using all or some of the difference values calculated at step 635. The impedance comparison value may be used for comparison with a threshold value to determine if the atomization element is wet or dry. As shown in FIG. 3, a positive slope within the frequency range applied to the vibratable element may be indicative of a dry atomization element. As such, determining when a positive slope is present (that is, measured impedance values increase as frequency increases) may be desired. To do this, various calculations may be performed. A calculation that may be performed to determine if impedance is increasing involves equations 2 and 3 and as detailed in relation to method 500.

At step 645, the impedance comparison value determined at step 640 may be compared to a threshold comparison value. Step 645 may be performed by a test module or a control module. This threshold comparison value may have been empirically determined. The same threshold value may be used for multiple nebulizer elements or may be specific to the nebulizer element that method 600 is being performed with. For example, a threshold comparison value may be selected that tends to be greater than impedance comparison values calculated for wet atomization elements, but less than impedance comparison values calculated for dry atomization elements. If an impedance comparison value is less than the threshold comparison value, the atomization element may be likely to be wet. If the impedance comparison value is greater than the threshold comparison value, the atomization element may be likely to be dry.

At step 650, if the comparison of step 645 indicates the impedance comparison value is greater than the threshold value, method 600 may proceed to step 655. At step 655, the vibratable element may stop being energized, such that the atomization element does not vibrate. This may be because the atomization element is expected to be dry. If method 600 proceeds to step 655, the vibratable element may not be energized until a user provides an indication that the vibratable element is to be energized again. In some embodiments, a period of time may be waited and the vibratable element may be reanalyzed to determine if wet or dry. This may be performed to determine if the determination that the atomization element was dry was due to one or more bubbles being present on the atomization element (which may have since dissipated or moved). If the atomization element is subsequently determined to be wet, method 600 may return to step 620. If the atomization element is again determined to be dry, the vibratable element may remain unenergized.

At step 650, if the comparison of step 645 indicates the impedance comparison value is less than the threshold value (e.g., the nebulizer element is likely wet), method 600 may return to step 620. At step 620, the vibratable element may be energized at one or more frequencies for atomizing a liquid by the atomization element, such as a liquid drug for a period of time, before performing the remaining steps of method 600 again. Method 600 may continue to be performed until the atomization element is determined to be dry and the vibratable element no longer energized, either to atomize liquid or to determine whether the atomization element is wet or dry.

Following method 600 being performed (and the second frequency range being established), the second frequency range may be used in the future to detect whether the atomization element is wet or dry. For example, this second frequency range may be stored by the nebulizer (e.g., the control module) and used in the field (e.g., outside of a manufacturing test environment). In some embodiments, outside of a manufacturing test environment, the nebulizer may return to using a wider frequency range, such as described in relation to method 500, when used in a post-manufacturing and post-test environment.

A computer system as illustrated in FIG. 7 may incorporate as part of the previously described computerized devices. For example, computer system 700 can represent some of the components of the test hardware or control module discussed in this application. FIG. 7 provides a schematic illustration of one embodiment of a computer system 700 that can perform at least portions of the methods provided by various embodiments, as described herein. It should be noted that FIG. 7 is meant only to provide a generalized illustration of various components, any or all of which may be utilized as appropriate. FIG. 7, therefore, broadly illustrates how individual system elements may be implemented in a relatively separated or relatively more integrated manner.

The computer system 700 is shown comprising hardware elements that can be electrically coupled via a bus 705 (or may otherwise be in communication, as appropriate). The hardware elements may include one or more processors 710, including without limitation one or more general-purpose processors and/or one or more special-purpose processors (such as digital signal processing chips, graphics acceleration processors, and/or the like); one or more input devices 715, which can include without limitation a mouse, a keyboard, and/or the like; and one or more output devices 720, which can include without limitation a display device, a printer, and/or the like.

The computer system 700 may further include (and/or be in communication with) one or more non-transitory storage devices 725, which can comprise, without limitation, local and/or network accessible storage, and/or can include, without limitation, a disk drive, a drive array, an optical storage device, a solid-state storage device, such as a random access memory (“RAM”), and/or a read-only memory (“ROM”), which can be programmable, flash-updateable and/or the like. Such storage devices may be configured to implement any appropriate data stores, including without limitation, various file systems, database structures, and/or the like.

The computer system 700 might also include a communications subsystem 730, which can include without limitation a modem, a network card (wireless or wired), an infrared communication device, a wireless communication device, and/or a chipset (such as a Bluetooth™ device, an 802.11 device, a WiFi device, a WiMax device, cellular communication facilities, etc.), and/or the like. The communications subsystem 730 may permit data to be exchanged with a network (such as the network described below, to name one example), other computer systems, and/or any other devices described herein. In many embodiments, the computer system 700 will further comprise a working memory 735, which can include a RAM or ROM device, as described above.

The computer system 700 also can comprise software elements, shown as being currently located within the working memory 735, including an operating system 740, device drivers, executable libraries, and/or other code, such as one or more application programs 745, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a non-transitory computer-readable storage medium, such as the non-transitory storage device(s) 725 described above. In some cases, the storage medium might be incorporated within a computer system, such as computer system 700. In other embodiments, the storage medium might be separate from a computer system (e.g., a removable medium, such as a compact disc), and/or provided in an installation package, such that the storage medium can be used to program, configure, and/or adapt a general purpose computer with the instructions/code stored thereon. These instructions might take the form of executable code, which is executable by the computer system 700 and/or might take the form of source and/or installable code, which, upon compilation and/or installation on the computer system 700 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer system (such as the computer system 700) to perform methods in accordance with various embodiments of the invention. According to a set of embodiments, some or all of the procedures of such methods are performed by the computer system 700 in response to processor 710 executing one or more sequences of one or more instructions (which might be incorporated into the operating system 740 and/or other code, such as an application program 745) contained in the working memory 735. Such instructions may be read into the working memory 735 from another computer-readable medium, such as one or more of the non-transitory storage device(s) 725. Merely by way of example, execution of the sequences of instructions contained in the working memory 735 might cause the processor(s) 710 to perform one or more procedures of the methods described herein.

The terms “machine-readable medium” and “computer-readable medium,” as used herein, refer to any medium that participates in providing data that causes a machine to operate in a specific fashion. In an embodiment implemented using the computer system 700, various computer-readable media might be involved in providing instructions/code to processor(s) 710 for execution and/or might be used to store and/or carry such instructions/code. In many implementations, a computer-readable medium is a physical and/or tangible storage medium. Such a medium may take the form of a non-volatile media or volatile media. Non-volatile media include, for example, optical and/or magnetic disks, such as the non-transitory storage device(s) 725. Volatile media include, without limitation, dynamic memory, such as the working memory 735.

Common forms of physical and/or tangible computer-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, EPROM, a FLASH-EPROM, any other memory chip or cartridge, or any other medium from which a computer can read instructions and/or code.

Various forms of computer-readable media may be involved in carrying one or more sequences of one or more instructions to the processor(s) 710 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 700.

The communications subsystem 730 (and/or components thereof) generally will receive signals, and the bus 705 then might carry the signals (and/or the data, instructions, etc. carried by the signals) to the working memory 735, from which the processor(s) 710 retrieves and executes the instructions. The instructions received by the working memory 735 may optionally be stored on a non-transitory storage device 725 either before or after execution by the processor(s) 710.

While a wide variety of drugs, liquids, liquid drugs, and drugs dissolved in liquid may be aerosolized, the following provides extensive examples of what may be aerosolized. Additional examples are provided in U.S. application Ser. No. 12/341,780, the entire disclosure of which is incorporated herein for all purposes. Nearly any anti-gram-negative, anti-gram-positive antibiotic, or combinations thereof may be used. Additionally, antibiotics may comprise those having broad spectrum effectiveness, or mixed spectrum effectiveness. Antifungals, such as polyene materials, in particular, amphotericin B, are also suitable for use herein. Examples of anti-gram-negative antibiotics or salts thereof include, but are not limited to, aminoglycosides or salts thereof. Examples of aminoglycosides or salts thereof include gentamicin, amikacin, kanamycin, streptomycin, neomycin, netilmicin, paramycin, tobramycin, salts thereof, and combinations thereof. For instance, gentamicin sulfate is the sulfate salt, or a mixture of such salts, of the antibiotic substances produced by the growth of Micromonospora purpurea. Gentamicin sulfate, USP, may be obtained from Fujian Fukang Pharmaceutical Co., LTD, Fuzhou, China. Amikacin is typically supplied as a sulfate salt, and can be obtained, for example, from Bristol-Myers Squibb. Amikacin may include related substances such as kanamicin.

Examples of anti-gram-positive antibiotics or salts thereof include, but are not limited to, macrolides or salts thereof. Examples of macrolides or salts thereof include, but are not limited to, vancomycin, erythromycin, clarithromycin, azithromycin, salts thereof, and combinations thereof. For instance, vancomycin hydrochloride is a hydrochloride salt of vancomycin, an antibiotic produced by certain strains of Amycolatopsis orientalis, previously designated Streptomyces orientalis. Vancomycin hydrochloride is a mixture of related substances consisting principally of the monohydrochloride of vancomycin B. Like all glycopeptide antibiotics, vancomycin hydrochloride contains a central core heptapeptide. Vancomycin hydrochloride, USP, may be obtained from Alpharma, Copenhagen, Denmark.

In some embodiments, the composition comprises an antibiotic and one or more additional active agents. The additional active agent described herein includes an agent, drug, or compound, which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system.

Examples of additional active agents include, but are not limited to, anti-inflammatory agents, bronchodilators, and combinations thereof.

Examples of bronchodilators include, but are not limited to, beta-agonists, anti-muscarinic agents, steroids, and combinations thereof. For instance, the steroid may comprise albuterol, such as albuterol sulfate.

Active agents may comprise, for example, hypnotics and sedatives, psychic energizers, tranquilizers, respiratory drugs, anticonvulsants, muscle relaxants, antiparkinson agents (dopamine antagnonists), analgesics, anti-inflammatories, antianxiety drugs (anxiolytics), appetite suppressants, antimigraine agents, muscle contractants, additional anti-infectives (antivirals, antifungals, vaccines) antiarthritics, antimalarials, antiemetics, anepileptics, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosphonates, respiratory syncytial virus antibody, cystic fibrosis transmembrane regulator (CFTR) gene, deoxyreibonuclease (Dnase), bactericidal/permeability increasing protein (BPI), anti-CMV antibody, 1 3-cis retinoic acid, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxacin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, teicoplanin, rampolanin, mideplanin, colistin, daptomycin, gramicidin, colistimethate, polymixins such as polymixin B, capreomycin, bacitracin; penems, such as penicillins including penicillinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefinetazole, ceftazidime, loracarbef, and moxalactam, monobactams like aztreonam; and carbapenems such as imipenem, meropenem; and agents of other classes, such as pentamidine isethionate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments, derivatives, and analogs thereof.

Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense. Further, an active agent may comprise live attenuated or killed viruses suitable for use as vaccines. Other useful drugs include those listed within the Physician's Desk Reference (most recent edition), which is incorporated herein by reference in its entirety.

The amount of antibiotic or other active agent in the pharmaceutical formulation will be that amount necessary to deliver a therapeutically or prophylactically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1 wt % to about 99 wt %, such as from about 2 wt % to about 95 wt %, or from about 5 wt % to 85 wt %, of the active agent, and will also depend upon the relative amounts of additives contained in the composition. The compositions of the invention are particularly useful for active agents that are delivered in doses of from 0.001 mg/day to 100 mg/day, such as in doses from 0.01 mg/day to 75 mg/day, or in doses from 0.10 mg/day to 50 mg/day. It is to be understood that more than one active agent may be incorporated into the formulations described herein and that the use of the term “agent” in no way excludes the use of two or more such agents.

Generally, the compositions are free of excessive excipients. In one or more embodiments, the aqueous composition consists essentially of the anti-gram-negative antibiotic, such as amikacin, or gentamicin or both, and/or salts thereof and water.

Further, in one or more embodiments, the aqueous composition is preservative-free. In this regard, the aqueous composition may be methylparaben-free and/or propylparaben-free. Still further, the aqueous composition may be saline-free.

In one or more embodiments, the compositions comprise an anti-infective and an excipient. The compositions may comprise a pharmaceutically acceptable excipient or carrier which may be taken into the lungs with no significant adverse toxicological effects to the subject, and particularly to the lungs of the subject. In addition to the active agent, a pharmaceutical formulation may optionally include one or more pharmaceutical excipients which are suitable for pulmonary administration. These excipients, if present, are generally present in the composition in amounts sufficient to perform their intended function, such as stability, surface modification, enhancing effectiveness or delivery of the composition or the like. Thus, if present, excipient may range from about 0.01 wt % to about 95 wt %, such as from about 0.5 wt % to about 80 wt %, from about 1 wt % to about 60 wt %. Preferably, such excipients will, in part, serve to further improve the features of the active agent composition, for example by providing more efficient and reproducible delivery of the active agent and/or facilitating manufacturing. One or more excipients may also be provided to serve as bulking agents when it is desired to reduce the concentration of active agent in the formulation.

For instance, the compositions may include one or more osmolality adjuster, such as sodium chloride. For instance, sodium chloride may be added to solutions of vancomycin hydrochloride to adjust the osmolality of the solution. In one or more embodiments, an aqueous composition consists essentially of the anti-gram-positive antibiotic, such as vancomycin hydrochloride, the osmolality adjuster, and water.

Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination.

Exemplary protein excipients include albumins such as human serum albumin (HSA), recombinant human albumin (rHA), gelatin, casein, hemoglobin, and the like. Suitable amino acids (outside of the dileucyl-peptides of the invention), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like.

The pharmaceutical formulation may also comprise a buffer or a pH adjusting agent, typically a salt prepared from an organic acid or base. Representative buffers comprise organic acid salts of citric acid, ascorbic acid, gluconic acid, carbonic acid, tartaric acid, succinic acid, acetic acid, or phthalic acid, Tris, tromethamine hydrochloride, or phosphate buffers.

The pharmaceutical formulation may also include polymeric excipients/additives, e.g., polyvinylpyrrolidones, celluloses and derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-beta-cyclodextrin and sulfobutylether-beta-cyclodextrin), polyethylene glycols, and pectin.

The pharmaceutical formulation may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations). Other pharmaceutical excipients and/or additives suitable for use in the compositions according to the invention are listed in “Remington: The Science & Practice of Pharmacy”, 19th ed., Williams & Williams, (1995), and in the “Physician's Desk Reference”, 52nd ed., Medical Economics, Montvale, N.J. (1998), both of which are incorporated herein by reference in their entireties.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered. Accordingly, the above description does not bound the scope of the claims.

1. A nebulizer, comprising:
a nebulizer element comprising an atomization element and a vibratable element, the vibratable element is configured to vibrate to cause the atomization element to atomize a liquid in contact with the atomization element;
a reservoir configured to hold the liquid that is to be supplied to the atomization element; and
a control module configured to:
output an electrical signal at an atomization frequency to energize the vibratable element;
vary a frequency of the electrical signal across a measurement frequency range to energize the vibratable element, wherein the measurement frequency range is from a first frequency to a second frequency;
while the vibratable element is being energized with the electrical signal that varies from the first frequency to the second frequency, measure a sequence of impedance values of the vibratable element; and
analyze the sequence of impedance values to determine if the atomization element is dry.

2. (canceled)

3. The nebulizer of claim 1, wherein the control module is further configured to:
if the atomization element is determined to not be in contact with the liquid, cease outputting the electrical signal to energize the vibratable element.

4. The nebulizer of claim 1, wherein the control module being configured to analyze the sequence of impedance values of the vibratable element to determine if the atomization element is dry comprises the control module being configured to:
analyze an amount of change among impedance values of the sequence of impedance values.

5. The nebulizer of claim 1, wherein the control module being configured to analyze the sequence of impedance values of the vibratable element to determine if the atomization element is dry comprises the control module being configured to:
calculate a sequence of difference values that indicates differences between at least some consecutive impedance values of the sequence of impedance values.

6. The nebulizer of claim 5, wherein the control module being configured to analyze the sequence of impedance values of the vibratable element to determine if the atomization element is dry comprises the control module being configured to:
calculate an impedance comparison value using the sequence of difference values; and
compare the impedance comparison value to a predefined threshold comparison value to determine if the atomization element is dry.

7. The nebulizer of claim 6, wherein the control module being configured to calculate the impedance comparison value using the sequence of difference values comprises the control module being configured to:
for each positive difference value of the sequence of difference values, add a squared value of the positive difference value to the impedance comparison value; and
for each negative difference value of the sequence of difference values, add an absolute value of the negative difference value to the impedance comparison value.

8. The nebulizer of claim 1, wherein:
the first frequency is lower than the second frequency; and
the control module being configured to output the electrical signal to energize the vibratable element comprises the control module being configured to output the electrical signal to energize the vibratable element of the nebulizer at multiple different frequencies between the first frequency and the second frequency.

9. The nebulizer of claim 8, wherein the first frequency is 95 kHz and the second frequency is 128 kHz.

10. The nebulizer of claim 1, wherein:
the control module being configured to output the electrical signal to energize the vibratable element comprises the electrical signal sweeping from the first frequency to the second frequency for less than 200 ms; and
the control module is configured to measure impedance values for the sequence of impedance values at a sampling interval of less than 5 ms.

11-13. (canceled)

14. A system, comprising the nebulizer of claim 1, the system further comprising:
a test module configured to:
energize the vibratable element while the atomization element is dry with a test electrical signal that sweeps a first frequency range, wherein the measurement frequency range defined by the first frequency and the second frequency is within the first frequency range and is smaller in bandwidth than the first frequency range.

15. The system of claim 14, wherein the test module is further configured to:
while energizing the vibratable element with the test electrical signal that sweeps the first frequency range, measure a test sequence of impedance values of the vibratable element; and
determine the first frequency and the second frequency at least partially based on the test sequence of impedance values.

16. The system of claim 14, wherein the control module of the nebulizer is further configured to store indications of the first frequency and the second frequency determined by the test module.

17. A method for determining an atomization element of a nebulizer is dry, the method comprising:
energizing a vibratable element of the nebulizer with an electrical signal that sweeps from a first frequency to a second frequency;
while energizing the vibratable element of the nebulizer with the electrical signal that varies from the first frequency to the second frequency, measuring a sequence of impedance values of the vibratable element of the nebulizer; and
analyzing the sequence of impedance values of the vibratable element of the nebulizer to determine if the atomization element of the nebulizer is dry.

18. The method for determining the atomization element of the nebulizer is dry of claim 17, the method further comprising:
energizing the vibratable element of the nebulizer at an atomization frequency to cause the atomization element to atomize liquid.

19. (canceled)

20. The method for determining the atomization element of the nebulizer is dry of claim 18, the method further comprising:
if the atomization element is determined to not be in contact with the liquid, cease energizing the vibratable element with the electrical signal.

21. The method for determining the atomization element of the nebulizer is dry of claim 20, the method further comprising:
after ceasing to energize the vibratable element with the electrical signal, waiting a period of time; and
after waiting the period time:
energizing the vibratable element of the nebulizer with the electrical signal that sweeps from the first frequency to the second frequency;
while energizing the vibratable element of the nebulizer with the electrical signal that varies from the first frequency to the second frequency, measuring a second sequence of impedance values of the vibratable element of the nebulizer; and
analyzing the second sequence of impedance values of the vibratable element of the nebulizer to determine if the atomization element of the nebulizer is dry.

22. The method for determining the atomization element of the nebulizer is dry of claim 17, wherein analyzing the sequence of impedance values of the vibratable element of the nebulizer to determine if the atomization element of the nebulizer is dry comprises:
analyzing an amount of change among impedance values of the sequence of impedance values.

23. The method for determining the atomization element of the nebulizer is dry of claim 17, wherein analyzing the sequence of impedance values of the vibratable element of the nebulizer to determine if the atomization element is dry comprises:
calculating a sequence of difference values that indicates differences between at least some consecutive impedance values of the sequence of impedance values.

24. The method for determining the atomization element of the nebulizer is dry of claim 23, wherein analyzing the sequence of impedance values of the vibratable element of the nebulizer to determine if the atomization element of the nebulizer is dry comprises:
calculating an impedance comparison value using the sequence of difference values; and
comparing the impedance comparison value to a predefined threshold comparison value to determine if the atomization element is wet or dry.

25. The method for determining the atomization element of the nebulizer is dry of claim 24, wherein calculating the impedance comparison value using the sequence of difference values comprises:
for each positive difference value of the sequence of difference values, adding a squared value of the positive difference value to the impedance comparison value; and
for each negative difference value of the sequence of difference values, adding an absolute value of the negative difference value to the impedance comparison value.

26-28. (canceled)

29. The method for determining the atomization element of the nebulizer is dry of claim 17, wherein the method is performed at periodic intervals while a liquid is being atomized using the atomization element of the nebulizer.

30. The method for determining the atomization element of the nebulizer is dry of claim 29, wherein consecutive periodic intervals of the periodic intervals are less than two seconds apart.

31. The method for determining the atomization element of the nebulizer is dry of claim 17, further comprising:
energizing the vibratable element while dry with a test electrical signal that sweeps a first frequency range, wherein a second frequency range defined by the first frequency and the second frequency is within the first frequency range and is smaller in bandwidth than the first frequency range.

32. The method for determining the atomization element of the nebulizer is dry of claim 31, further comprising:
while energizing the vibratable element with the test electrical signal that sweeps the first frequency range, measuring a test sequence of impedance values of the vibratable element of the nebulizer; and
determining the first frequency and the second frequency at least partially based on the test sequence of impedance values.

33-40. (canceled)

41. A method for delivering a medicament to a patient, the method comprising:
providing a nebulizer comprising a housing defining a mouthpiece and having an atomization element and a vibratable element;
supplying a liquid medicament to the atomization element;
energizing the vibratable element of the nebulizer with an electrical signal at an atomization frequency causing the atomization element to atomize the liquid medicament, wherein
the atomized liquid medicament is available for inhalation through the mouthpiece;
varying the electrical signal across a measurement frequency range that sweeps from a first frequency to a second frequency;
while sweeping the electrical signal from the first frequency to the second frequency, measuring a sequence of impedance values of the vibratable element of the nebulizer; and
analyzing the sequence of impedance values of the vibratable element of the nebulizer to determine the atomization element is dry of the liquid medicament; and
ceasing to energize the vibratable element with the electrical signal at least partially based on determining the atomization element is dry of the liquid medicament.
